(19) United States
(12) Patent Application Publication
(10) Pub. No.: US 2007/0100384 A1
(43) Pub. Date: May 3, 2007

(54) Implantable medical system with long range telemetry

(76) Inventors: David R. Fischell, Fair Haven, NJ (US); Gregg Turi, Mount Olive, NJ (US); Steven R. Johnson, Fair Haven, NJ (US)

Correspondence Address: ROSENBERG, KLEIN & LEE, 3458 ELLICOTT CENTER DRIVE-SUITE 101, ELLICOTT CITY, MD, 21043, US

(21) Appl. No.: 11/635,623

(22) Filed: Dec. 8, 2006

Related U.S. Application Data
(62) Division of application No. 10/994,466, filed on Nov. 23, 2004.
(60) Provisional application No. 60/524,077, filed on Nov. 24, 2003.

(51) Int. Cl. A61N 1/36

(52) U.S. Cl. 607/32

(57) ABSTRACT

An implantable medical system for implantation within the body of a patient is provided. The system includes an implanted device having an implant casing and a long range telemetry sub-system housed therein. The system also includes an implantable lead operationally coupled to the implanted device and an antenna coupled to the implant casing to extend therefrom. The antenna is operationally coupled to the long-range telemetry sub-system to enable wireless bi-directional communication between the long range telemetry sub-system and predetermined external equipment disposed outside the body of the patient.

### RELATED APPLICATIONS

This application is a Divisional Patent Application of co-pending application Ser. No. 10/994,466, filed on 23 Nov. 2004.

### FIELD OF THE INVENTION

This invention relates to the field of medical devices implantable within a human patient and having the ability to communicate with remote equipment.

### BACKGROUND OF THE INVENTION

Known implantable devices including pacemakers and Implantable Cardiac Defibrillators (ICDs) include the capability to communicate with remote equipment while implanted in the body of a patient. Such communication occurs by means of an antenna of the remote equipment placed within inches of the implant to be able to reliably send and receive data to/from that implant. Recently, Biotronik Company has developed a long range telemetry system for pacemakers that allows data from the pacemakers to be transmitted to a remote receiver disposed several meters away from the patient. However, the Biotronik long range data communication system is unidirectional, meaning that data are transmitted in one direction, e.g., only from the implant to the remote receiver. This precludes error checking and such other useful functions as device programmability without employing a separate near field antenna.

U.S. Pat. No. 6,609,023 issued to Fischell, et al., describes a two-way long range data communication system for data transmission between an implanted cardiac event detection system and remote equipment in both directions. In the Fischell, et al.'s system, data transmission from the remote equipment to the implant is enabled by the implant turning ON its telemetry sub-system at regular intervals to “listen” for the data to be received. Such telemetry sub-systems, for example, the CC1000 chipset from CHIPCOM, consume significant power during the “listening” phase of their operation. In order to save power and to extend the operational life of the implant, such intervals for “listening” for the data to be received are kept on the order of 30 seconds or longer. This precludes fast time response of the implanted devices to commands from the remote equipment.

Since a telemetry antenna is an essential part of a medical implantable system, specific arrangements have been developed to improve the operational characteristics of implantable medical devices. For example, U.S. Pat. No. 5,342,408 describes a telemetry system for an implantable cardiac device in which a telemetry antenna is placed in a plastic header of an implanted cardiac device to facilitate high speed communication between external equipment, such as an external programmer, and the cardiac implant device. Another U.S. Pat. No. 5,456,698 describes a pacemaker in which a telemetry antenna is placed in a plastic outer casing (or “shroud”) of the implant. Yet another arrangement is shown in U.S. Pat. No. 6,614,406 wherein an antenna is placed in an antenna compartment made of a dielectric material extending from the header to wrap circumferentially around a curved portion of the device housing. U.S. Pat. Nos. 4,543,955 and 5,058,581 describe body implantable devices which use their respective leads as a telemetry antenna for the implantable device.

None of the above arrangements is ideal, for the placement of a telemetry antenna in the header or a plastic outer casing of the implant limits the usable antenna length, while employing a lead of the implant as the antenna causes RF energy to be delivered into the heart. Although the Fischell's AMI detection implant described in U.S. Pat. No. 6,609,023 only requires a unipolar lead, the device is typically implanted with a standard bipolar pacemaker lead so that if a pacemaker is needed by the patient, no new lead needs to be implanted. Using a second conductor in the lead as the antenna is not desirable as it also has the negative effect associated with delivering RF energy into the heart.

It would be therefore highly desirable to realize an implantable medical system free of these and other shortcomings of prior implantable devices.

### SUMMARY OF THE INVENTION

It is therefore an object of the present invention to provide a system including an implanted medical device (implant) and an external transceiver between which long range two-way data communication may be effected. In accordance with the present invention, factors such as antenna location, antenna configuration, and remote electromagnetic signaling to the implant to turn ON/OFF the long range telemetry sub-system are advantageously combined.

It is another object of this invention to provide an implantable medical device with long range telemetry where the antenna is positioned in a connecting cable located between a device casing and an implantable lead of the device.

An additional object of the present invention is to provide an implantable medical device with long range telemetry where an antenna may be located outside of the device casing and provided with its own feed through the casing.

Still another object of this invention is to provide an implantable medical device with long range telemetry where the antenna is located in close proximity (outside, inside or within) to a window made from a non-conductive material placed in the casing of the implant.

Yet another object of the present invention is to provide an implantable medical device with long range telemetry where the device includes a magnetic switch function to enable two-way long range telemetry.

Yet another object of the present invention is to provide an implantable medical device with long range telemetry where the device has a near field (<200KHz) electromagnetic sensor for enabling two-way long range telemetry when external equipment placed within 6 inches or closer proximity of the implanted device sends an appropriate low power electromagnetic signal to the implant.

It is an additional object of the present invention to provide an implantable medical device with long range telemetry where the implant has a near field (<200KHz) electromagnetic sensor for enabling two-way long range telemetry when external equipment located more than 6 inches away from the implant sends an appropriate high power electromagnetic signal.

Still another object of the present invention is to provide an implantable medical device having an antenna which is arranged as the proximal section of one of the conducting wires of a modified pacemaker lead and where a connecting module within the lead connects the proximal section of the wire to the distal section of the wire for use with a pacemaker or Implantable Cardiac Defibrillator (ICD).

In one embodiment of the antenna configuration, a multi-conductor connecting cable is located between a header of the implant casing and an implantable lead. In the connecting cable, one conductor is used as the antenna, while the other conductors are used for connection to the lead. The proximal end of such a connecting cable may either connect through feed-throughs directly to the electronic circuitry of the implant or attach to a standard implantable device header. The distal end of the connecting cable includes measures for connecting to an implantable lead.

In certain embodiments, such lead may be a standard bipolar pacemaker lead. One conductor of the connecting cable may then connect to one electrode of the bipolar lead, while other conductor may terminate within the connecting cable's length to serve as the antenna.

In an alternate embodiment, the antenna may be located outside of the casing of the implant. This may either be in the form of a loose wire, or a wire attached to a non-conducting casing extension.

In another embodiment of the antenna configuration, a non-conducting window is formed in a side wall of the implant casing with the antenna placed in close proximity to the window, the antenna may then be disposed inside the implant casing, within the window material, or attached to the window outside the implant casing.

Another alternate embodiment of the system of the present invention employs a modified bipolar pacemaker lead in which a wire connected to a ring electrode is normally discontinuous at a location part way down the lead. In this state, the wire has a proximal section and a distal section which are normally displaced each from the other. The proximal section of the wire is designed to function as an antenna for the implant's telemetry sub-system. A connecting module provides for connection between the proximal and distal sections of the wire to allow the bipolar pacemaker lead to function with a standard pacemaker. This concept is applicable to any wire within any lead. For example, the connectable lead wire concept may be implemented using the tip wire in a bipolar lead or using any one of the wires in an Implantable Cardiac Defibrillator (ICD) or dual chamber pacemaker lead.

To reduce power consumption in a “listening” mode of operation for receiving commands from a remote device, numerous different techniques may be used in the system of the present invention. These techniques include the use of magnetic switching, near field activation, or a remote high power signal burst activation.

The use of magnetic switching is perhaps the simplest of these techniques. Most implantable devices have a magnetic switch for switching ON and OFF specific functions. In this case, placement of a magnet near the implant would cause the device to turn ON the long range telemetry receiver for a preset period of time to “listen” for long range telemetry commands. After the preset period elapses, the receiver is turned OFF to save power. The receiver may remain ON continuously during the preset period or may alternatively be switched periodically (e.g. for 100 ms every 2 seconds) during the preset time period to provide even more efficient energy saving.

The near field activation technique uses a typical near field receive circuit, as is found in most pacemakers. In this case, an electromagnetic signal sent by a device held close to the implant's location will be received by the near field receive circuit which then triggers the implant to switch the long range telemetry ON for a preset period of time, either continuously or periodically.

The high power burst technique sends an electromagnetic signal burst from a device located six inches or more away from the implant. The signal has sufficient intensity when received by the near field receiver circuitry in the implant to activate the long range telemetry system, much as with the near field activation technique. Specific security codes may be included in the near field activation signal or high power burst signal to minimize or eliminate the chance of inadvertently activating the long range telemetry circuitry for other signal sources.

The antenna for the near field receive circuitry can either be the same as the long range telemetry antenna or it can be a separate antenna located within the header or within the casing of the implant.

These and other objects and advantages of the present invention will become apparent to a person of ordinary skill in this art upon reading of the detailed description of this invention including the associated drawings as presented herein.

### BRIEF DESCRIPTION OF THE DRAWINGS

FIG. 1 schematic diagram illustrating one system embodiment of the present invention, which includes an implanted portion and an external portion communicating each with the other;

FIG. 2 is a schematic block diagram of the system embodiment of an implant portion with long range telemetry in accordance with the present invention;

FIG. 3A is a side view of one embodiment of a connecting cable antenna formed in accordance with the present invention;

FIG. 3B partially cutaway side view of the embodiment shown in FIG. 3A illustrating a portion of the internal structure;

FIG. 4 shows the embodiment of the implanted portion of the system of the present invention shown in FIG. 3A, with the connecting cable antenna attached to a standard bipolar lead header connection;

FIG. 5 shows an alternate embodiment of the implanted portion of the system of the present invention, with a loose antenna located outside of the case of the implanted portion;

FIG. 6 shows another alternate embodiment of the implanted portion of the system of the present invention, with a window in the outer side of the implant casing and an antenna located in close proximity to the window;

FIG. 7A is a cross sectional view of the implanted portion of FIG. 6 with the antenna disposed outside of the window;

FIG. 7B is a cross sectional view of the implanted portion of FIG. 6 with the antenna disposed inside of the window;

FIG. 7C is a cross sectional view of the implanted portion of FIG. 6 with the antenna disposed within the window;

FIG. 8 shows an embodiment of the implanted portion of the system of the present invention with a permanently attached connecting cable antenna;

FIG. 9 shows an embodiment of the connecting cable with an attached subcutaneous lead;

FIG. 10 shows an embodiment of the antenna configuration using a connectable lead wire;

FIG. 11A is an enlarged view of the connection module within the lead of FIG. 10 shown in the open configuration;

FIG. 11B is an enlarged view of the connection module within the lead of FIG. 10 shown in the closed configuration;

FIG. 12A is the cross section of the connection module of FIG. 11A taken along line 12A-12A; and

FIG. 12B is the cross section of the connection module of FIG. 11B taken along line 12B-12B.

### DETAILED DESCRIPTION OF THE INVENTION

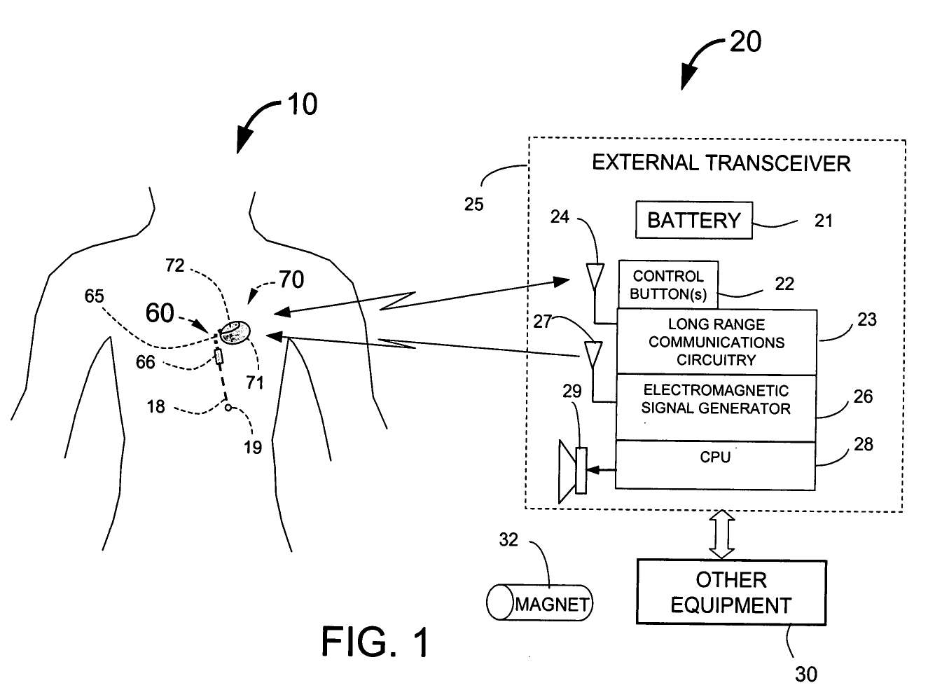

Regarding FIG. 1, an implantable medical system with long range telemetry of the present invention includes an implanted portion 10 and an external portion 20. The implanted portion 10 includes an implanted medical device (also referred to herein as an implant) 70 having a connecting cable 60 with a lead connector 66 that attaches to a lead 18 having electrode(s) 19. The implant 70 includes a casing 71 and a header 72. The implant 70 may be a diagnostic device with patient alerting capability such as described by Fischell, et al. in U.S. Pat. No. 6,609,023, or it may be a therapeutic device such as a pacemaker, Implantable Cardiac Defibrillator (ICD), an implantable drug pump, or the like.

The external portion 20 includes an external transceiver 25. A battery 21 may be embedded into the external transceiver 25 which is connected to other equipment 30. The other equipment 30 may include a physician's programmer and other display and command devices such as PDAs or cell phones. If power is provided by the other equipment 30 to the external transceiver 25 then no battery 21 is necessary. The external transceiver 25 also includes one or more control buttons 22, long range communications circuitry 23 provided with an antenna 24, and an electromagnetic signal generator 26 provided with an antenna 27. These components are managed by a CPU 28 having an acoustic transducer 29 coupled thereto.

A magnet 32 may also be included as a part of the external portion 20. The magnet 32 may be arranged as a separate part, or may alternatively be integrated into the external transceiver 25.

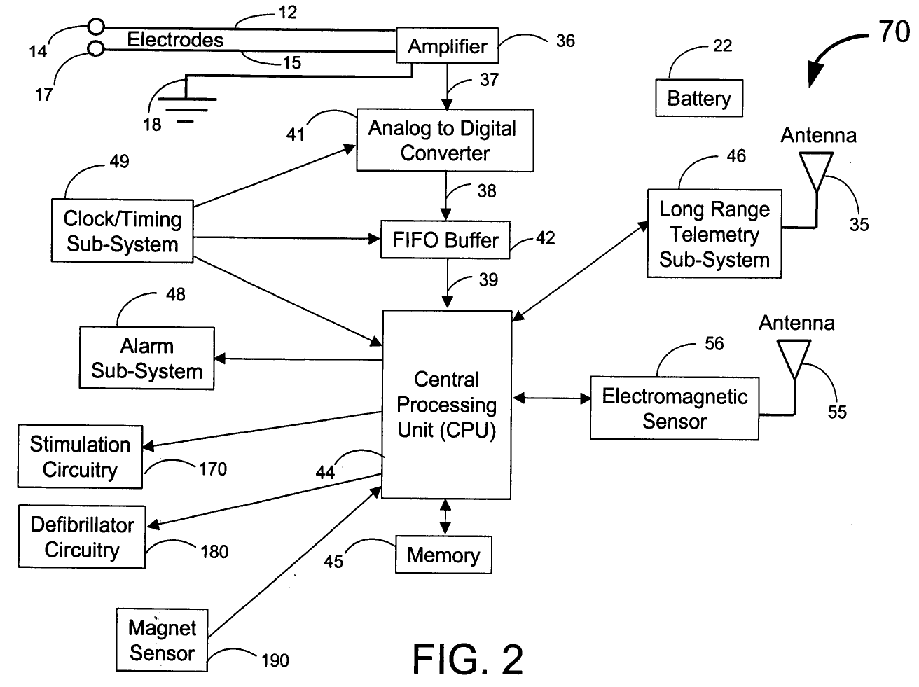

FIG. 2 is a schematic block diagram of the implanted medial device 70 which generally includes a battery 22, long range telemetry sub-system 46, and an antenna 35. A CPU 44 having a memory block 45, in conjunction with the clock/timing sub-system 49, controls the function of the implanted medical device 70. Incoming signals from electrodes 14, 17, and 19 are amplified by an amplifier 36, digitized by an analog-to-digital converter 41 and temporarily stored by a FIFO buffer 42. The implanted medical device 70 may also contain electrical stimulation circuitry 170 and/or cardiac defibrillator circuitry 180 coupled to the CPU 44 which are operable to deliver electrical stimulation to the heart through one or more electrodes, such as the electrodes 12 and 15.

Patient alerting is provided by the alarm sub-system 48 which may use vibrational, acoustic, electrical tickle or other suitable techniques to alert the patient to a specific event identified by the CPU 44.

A magnet sensor 190 permits triggering of device commands by placing the magnet 32 of FIG. 1 in close proximity to the implant 70. A near field electromagnetic sensor 56 with an antenna 55 is also present in the implant 70.

Current long range telemetry chip sets such as the Chipcom CC1000 chipset or the RF Microdevices Ash hybrid consume significant power even in the “listening” mode of operation of the implant 70. Consequently, the electromagnetic sensor 56 and/or magnet sensor 190 are the extremely important for efficient use of supplied power and significantly longer life of the battery 22 in the device of the present invention. Efficiency is heightened by an arrangement in which the long range telemetry sub-system 46 is normally turned OFF and only turned ON responsive to placement of the magnet 32 of FIG. 1 in close proximity to the magnet switch 190 or to detection of a specific signal by the electromagnetic sensor 56.

Once activated, the long range telemetry sub-system 46 operates to “listen” continuously or intermittently for a preset period. It listens for incoming long range data communication from the long range communications circuitry 23 of the external transceiver 25 shown in FIG. 1. If no signal is received, the long range telemetry sub-system 46 is turned OFF to save power. For maximum power conservation, the implanted medical device 70 may activate the electromagnetic sensor 56 only on a periodic basis. For example, the long range telemetry sub-system 46 might be turned ON to listen for ½ second every 5 seconds.

It is envisioned that the electromagnetic sensor 56 would be similar to the near field telemetry sub-systems present in current pacemakers and ICDs and would operate at frequencies below 200 KHz, for example, preferably in the range of 80-100 KHz. The antenna 55 may be of a suitable type known in the art, such as a simple inductive coil antenna used in current pacemakers.

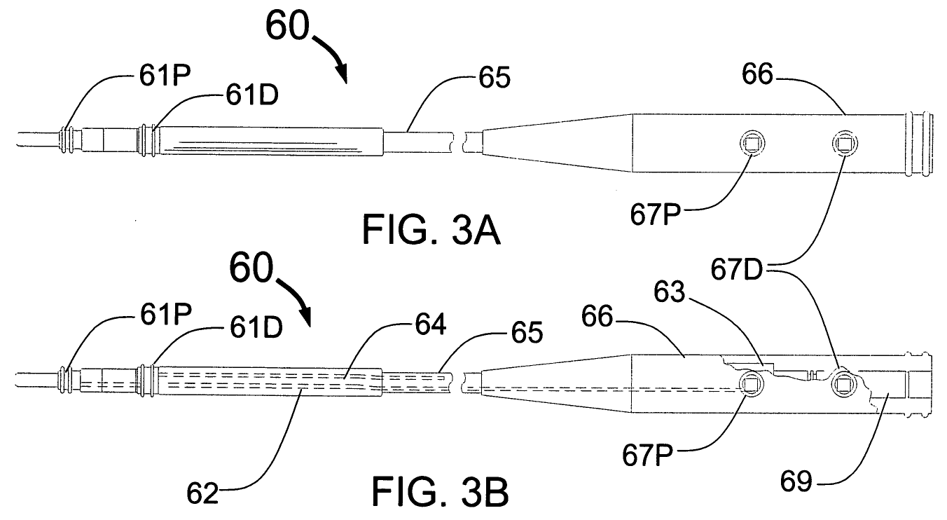

FIG. 3A is a side view of the connecting cable 60 having a distal ring 61D, proximal ring 61P, a cable 65 and a lead connector 66 with fastening screws 67D and 67P. The proximal end of the connecting cable 60 is designed to be attached to the header 72 of the implanted medical device 70 as shown in FIG. 1.

FIG. 3B illustrates the details of the internal structure of the connecting cable 60. The proximal ring 61P connects through a conductor 62 to a proximal contact 63 in the lead connector 66 of the connecting cable 60. The fastening screw 67P secures one of the conductors for a bipolar lead, such as the lead 18 of FIG. 1, against the proximal contact 63. The distal ring 61D connects to the antenna wire 64 which terminates at a predetermined distance from distal ring 61D in a manner appropriate for the frequencies of the signals to be transmitted and received by the antenna wire 64. In this embodiment, the antenna wire 64 acts as either or both of the antennas 35 and 55 shown in FIG. 2.

The proximal fastening screw 67P secures the proximal ring of an attachable bipolar lead to the proximal contact 63. Although no connection is shown between either of the rings 61D or 61P of the connecting cable 60 and the distal contact 69, it is envisioned that if a third ring is added to the distal end of the connecting cable 60, then both poles of a bipolar lead may be connected through to the implanted medical device 70 that would then require three contacts in its header 72 (one for the antenna wire 64 and two for both poles of a connected bipolar lead).

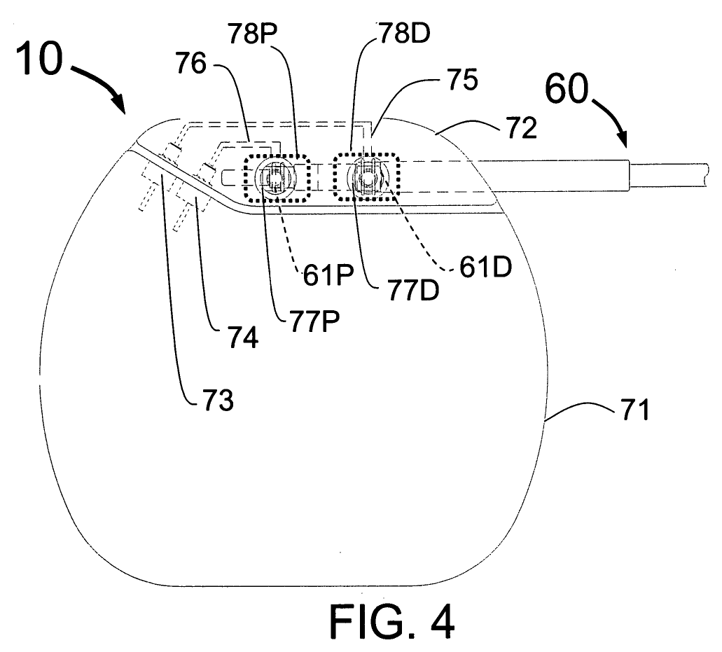

FIG. 4 shows the implanted portion 10 of the implantable system of the present invention where the connecting cable 60 couples to a standard bipolar lead header 72 attached to the casing 71 of the implant 70. As in most such implants, the conductors 75 and 76 in the header 72 connect to the electronics inside the casing 71 via the feed-throughs 73 and 74, respectively. The conductor 75 connects at its distal end to the contact 78D that will be pressed against the distal ring 61D of the connecting cable 60 when an adjustable member such as a set screw 77D is tightened. Similarly, the conductor 76 connects at its distal end to the contact 78P that will be pressed against the proximal ring 61 P of the connecting cable 60 when an adjustable member such as a screw 77P is tightened.

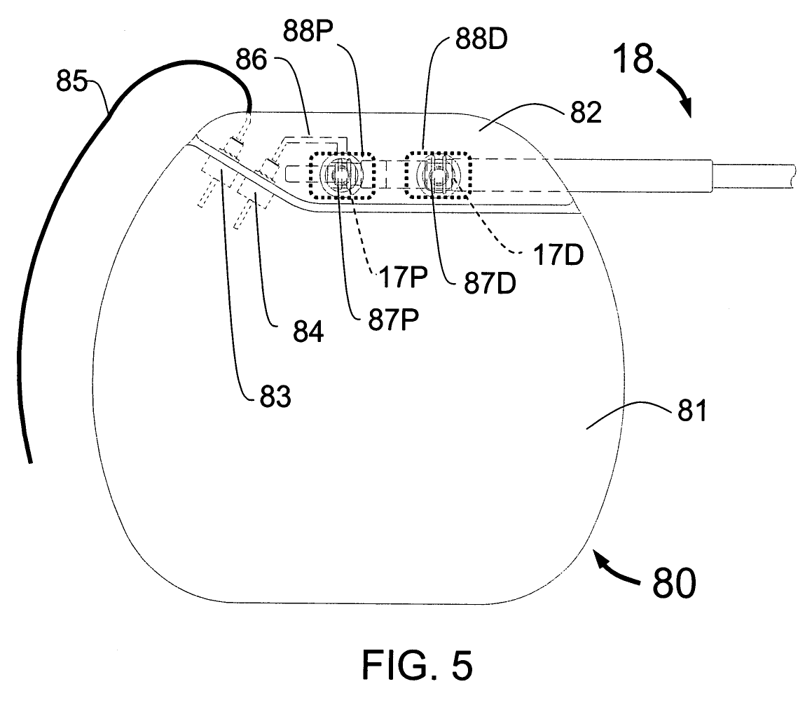

FIG. 5 illustrates an alternate embodiment of the implant 80 of the present invention wherein a loose antenna 85 located outside of a casing 81 is employed. The implant 80 includes the casing 81 and a header 82, and is coupled directly to a bipolar lead 18 with respective distal and proximal rings 17D and 17P. The loose antenna 85 connects through the header 82 and a feed through 83 to circuitry inside the casing 81 of the implant 80. A conductor 86 connects a contact 88P to a feed through 84 which also connects to circuitry inside the casing 81 of the implant 80. A set screw 87P is provided as shown, which when tightened, presses the contact 88P against the proximal ring 17P of the lead 18. Another set screw 87D is provided as shown, which when tightened, presses a contact 88D against the distal ring 17D of the lead 18. Although, in FIG. 5, the contact 88D is not connected by a feed through to circuitry inside the casing 81 of the implant 80, a third feed through and conductor may be added for this contact in accordance with another aspect of the present invention.

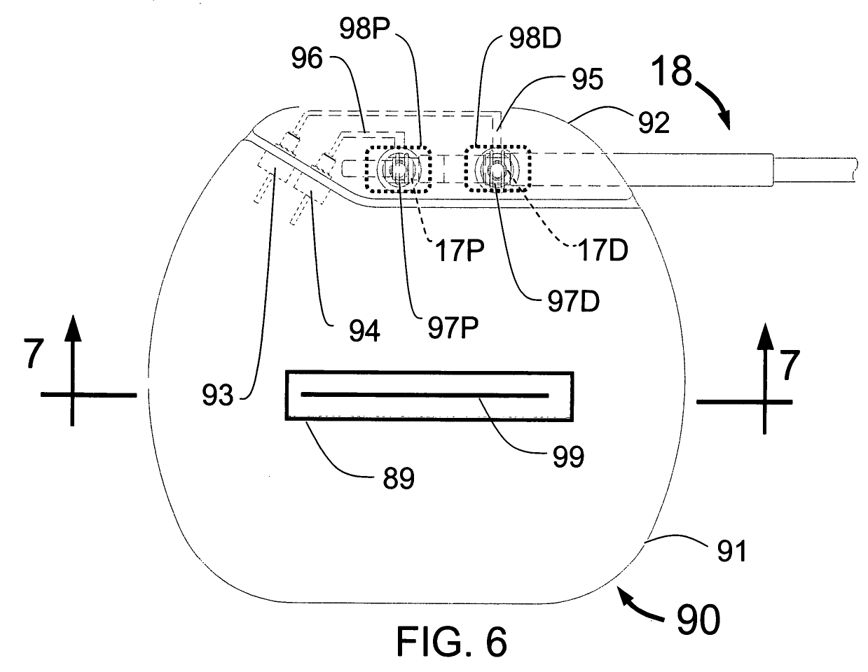

FIG. 6 illustrates still another embodiment of the implant 90 of the present invention with a window 89 made of non-conducting material disposed at an outer side of the implant casing 91. An antenna 99 is located in close proximity to the window 89. The implant 90 with the casing 91 and a header 92 connects directly to a bipolar lead 18 with respective distal and proximal rings 17D and 17P. A conductor 96 connects a contact 98P to a feed through 94 which connects to circuitry inside the casing 91 of the implant 90. A set screw 97P is provided, which when tightened, presses the contact 98P against the proximal ring 17P of the lead 18. Likewise, a conductor 95 connects a contact 98D to a feed through 93 which also connects to circuitry inside the casing 91 of the implant 90. A set screw 97D is provided, which when tightened, presses the contact 98D against the proximal ring 17D of the lead 18.

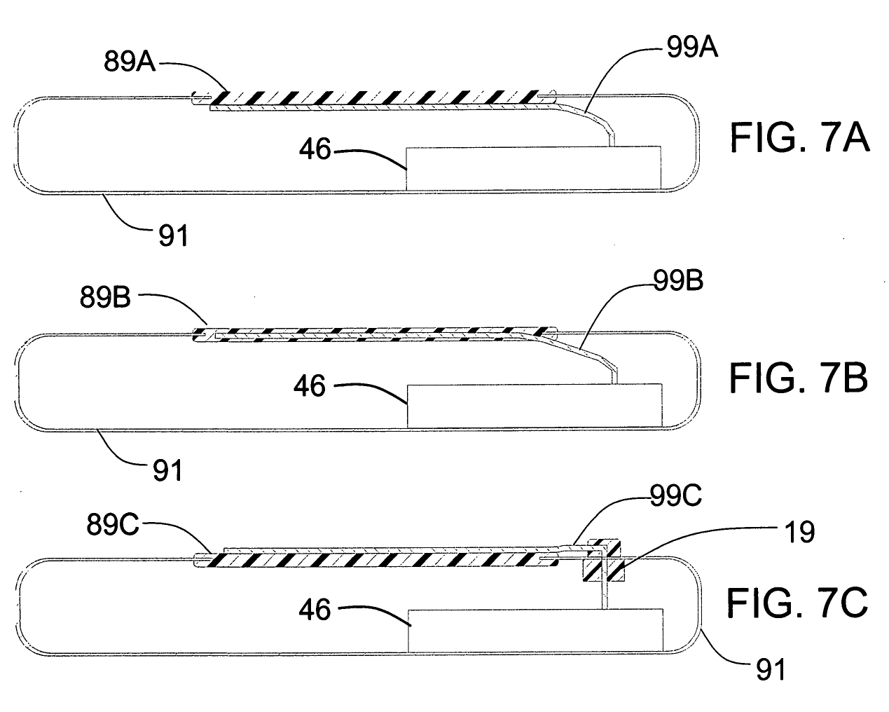

FIG. 7A is a cross sectional view of an embodiment of the implant 90 shown in FIG. 6, taken along line 7-7 thereof. In this particular embodiment, the window 89A is formed as shown in a side wall of the casing 91. The antenna 99A is positioned inside of that window 89A, bearing against an inner surface thereof. The antenna 99A connects to extend from the long range telemetry circuitry 46.

FIG. 7B is a cross sectional view of another embodiment of the implant 90 shown in FIG. 6, taken along line 7-7 thereof. In this alternate embodiment, the window 89B is again formed in a side wall of the casing 91. The antenna 99B is positioned within that window 89B to be at least partially embedded therein. The antenna 99B connects to extend from the long range telemetry circuitry 46.

FIG. 7C is a cross sectional view of still another embodiment of the implant 90 shown in FIG. 6 taken along line 7-7 thereof. Here, the antenna 99C is disposed outside of the window 89C which is, again, formed in a sidewall of the casing 91. The antenna 99C extends along an outer surface of the window 89C, and connects by a feed-through 19 formed in the casing 91 to the long range telemetry circuitry 46.

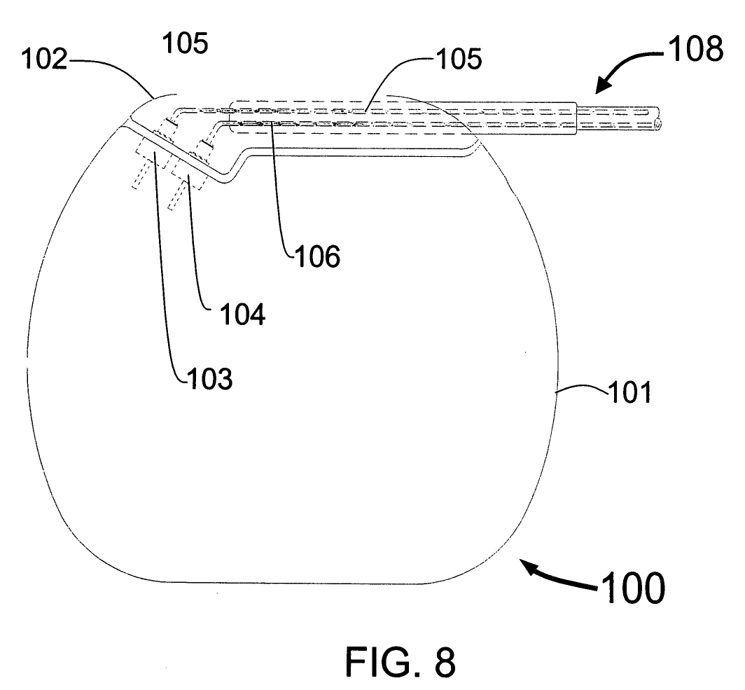

FIG. 8 illustrates yet another embodiment of the implanted system 100 of the present invention wherein an integrated connecting cable 108 is employed. The implant 100 includes a casing 101 and a header 102. An antenna 105 is provided within the integrated connecting cable 108 itself, and connects via a feed-through 103 to circuitry inside the case 101. A proximal end of the connecting cable 108 may be formed much like that of the connecting cable 60 shown in FIGS. 3A and 3B. A proximal end of the conductor 106 connects to the lead contact 63 at the proximal section of the integrated connecting cable 108 in much the same manner that conductor 62 does in FIG. 3B. A distal end of the conductor 106 connects via a feed-through 104 to circuitry inside the casing 101 of the implant 100. An advantage of this implant 100 embodiment over the embodiments employing attachable connecting cables is that the integrated connecting cable 108 affords a smaller header 102 than the connectable connecting cable 60 of FIGS. 3A, 3B, and 4, for instance. This allows for either a smaller overall implant or for increased space within the casing to accommodate device electronics and battery.

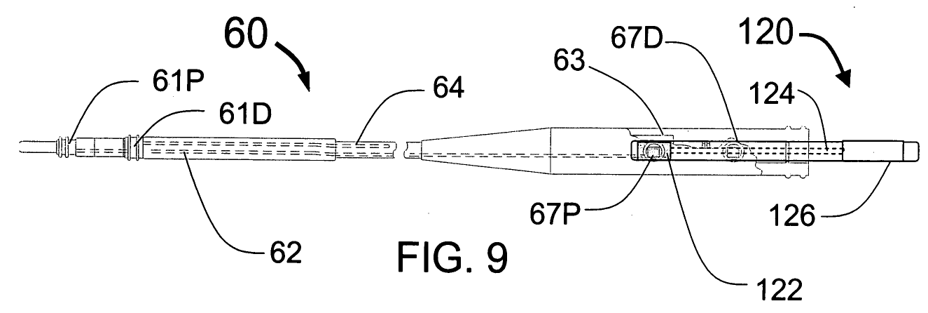

FIG. 9 shows the connecting cable 60 of FIGS. 3A, 3B, and 4 formed with an attached subcutaneous lead 120, the subcutaneous lead 120 preferably includes a conductor 124 which connects a distal ring 122 with an electrode 126.

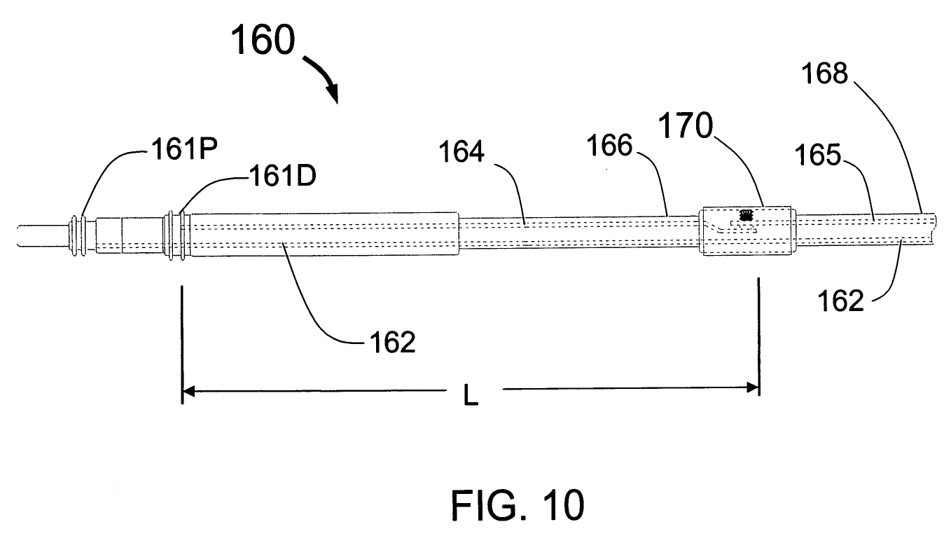

FIG. 10 shows a modified bipolar lead 160 formed in accordance with yet another embodiment of the present invention. The bipolar lead 160 preferably includes a standard proximal end with proximal ring 161P and distal ring 161D. The proximal ring 161P is connected to a wire 162 whose free end terminates at a tip electrode (not shown) for the bipolar lead 160. The distal ring 161D is connected to a proximal conducting wire 164 whose free end terminates at a connecting module 170. A distal connecting wire 165 extends from the connecting module 170 to terminate at a ring electrode (not shown) for the bipolar lead 160. Known distal tip configurations of bipolar leads include those manufactured and sold by St. Jude Medical, Guidant or Medtronic.

The connecting module 170 preferably serves to connect a proximal lead body 166 and a distal lead body 168. In the configuration of FIG. 10, the proximal wire 164 is detached from the distal wire 165. This allows the proximal wire 164 to function as an antenna of length “L” for an implanted device such as the device of FIG. 4. The proximal wire 164 has a length “L” that is preferably optimized for operation in the particular RF communication frequency range intended for the implanted device's telemetry sub-system 46, such as shown in FIG. 2. The length L is preferably set between 1 and 6 inches in approximate length. An advantage of the embodiment illustrated in FIG. 10 is that there is no need for a separate multi-wire connecting cable 60 of the type shown in FIG. 9 to avoid delivering energy from the antenna into the heart. The embodiment of FIG. 10 also allows reconfiguration of the lead 160 to serve as a bipolar lead adapted for a pacemaker or ICD. Although this embodiment is shown for a bipolar lead having two electrodes, a connecting module 170 may be used in accordance with alternate embodiments of the present invention to accommodate a lead having three or more electrodes.

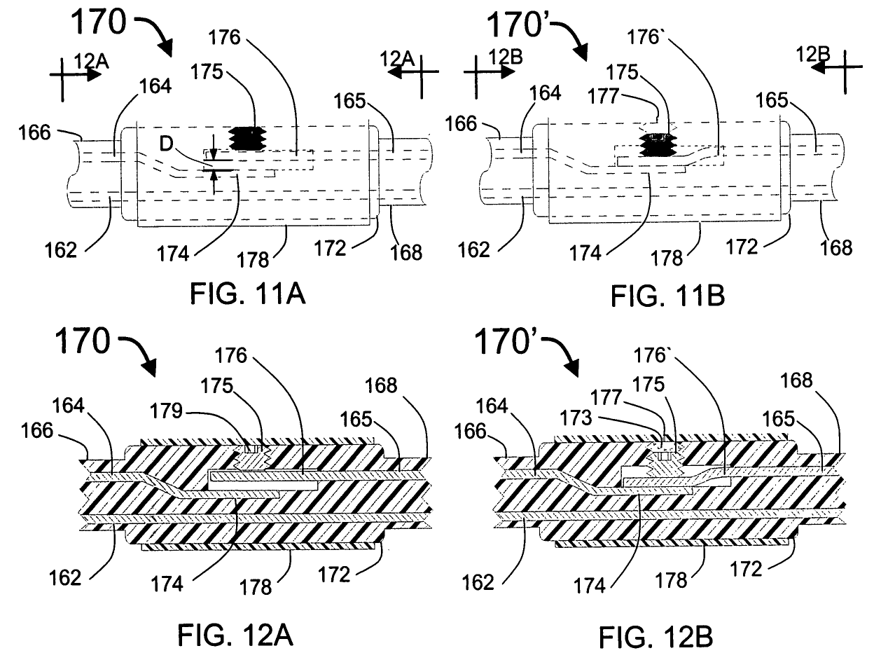

FIG. 11A shows on an enlarged scale the connection module 170 of FIG. 10 connecting the proximal lead body 166 and distal lead body 168. The wire 162 passes through the connection module 170. The connection module 170 preferably include a main body 172, an elastomer sealing sheath 178 and a set screw 175. The set screw may be advanced using any suitable means known in the art, such as an Allen (hex) type wrench (not shown). In FIG. 11A, a distal end 174 of the proximal wire 164 and a proximal end 176 of the distal wire 165 are, detached from each other separated by a distance “D.”
FIG. 11B shows on an enlarged scale the connection module 170′ of FIG. 10 upon reconfiguration of the lead 160 to a bipolar lead configuration. The reconfiguration utilizes suitable connecting measures for connecting the proximal wire 164 to the distal wire 165. Suitable connecting measures may be used, for example, to reconfigure the lead in the following manner:
1. cutting the elastomer sealing sheath with a scalpel to produce the slit 177;
2. inserting an Allen (hex) or other suitable type wrench through the slit to engage set screw 175 or other adjustable member (by insert into a hexagonal opening formed in a top of the set screws, for instance);
3. advancing the set screw to depress the proximal end 176′ of the distal wire 165 so that it contacts the distal end 174 of the proximal wire 164; and,
4. resealing the elastomer sealing sheath 178 using silicone or another plastic substance that will harden after injection through the slit 177.

Suitable variations of these measures may be employed. For instance, instead of cutting and resealing the sheath 178, a self-sealing slit may be used.

FIG. 12A is a cross sectional view of the connection module 170 of FIG. 11A taken along line 12A-12A thereof and showing the junction of the proximal lead body 166 and the distal lead body 168. The wire 162 passes through the connection module 170. The connection module 170 preferably includes a main body 172, an elastomer sealing sheath 178 and a set screw 175. The set screw is preferably formed with a hexagonal opening 179 at its top. The distal end 174 of the proximal wire 164 and the proximal end 176 of the distal wire 165 in this configuration remain detached from each other.

FIG. 12B is a cross sectional view of the connection module 170′ of FIG. 11A taken along line 12A-12A thereof upon reconfiguration of the lead 160 to its bipolar lead configuration. As described, the reconfiguration involves a connecting measure for connecting the proximal wire 164 to the distal wire 165. The final step (4) in the illustrative reconfiguration process described results in resealing the elastomer sealing sheath 178 with preferably a plastic substance 173 which substantially fills the slit above the set screw 175.

While the set screw 175 mechanically pushes the proximal end 176 of the distal wire 165 to make or break connection with the distal end 174 of the proximal wire 164, other alternate techniques may be employed in accordance with the present invention. For example, turning the set screw may extend a telescopic piece that connects the ends 174 and 176. Another alternate mechanism may be of the “fastener” type often used in assembling shelving units where one half turn locks or unlocks the “fastened” connection.

In accordance with yet another alternate embodiment of the present invention, an indicator may be used to show the state (connected or detached) of the lead wire connection. Such an indicator may change color, much as in the strip closures used in plastic bags, or may employ specific marks to visually indicate the state of lead wire connection.

Although FIGS. 10-12B illustrate a specific exemplary arrangement for pushing together the proximal and distal wires 164 and 165, numerous other suitable arrangements may be employed in accordance with the present invention. For example, a system may be used where a predefined rotational motion causes the wires to align and connect. In another example, a predefined bending motion causes the wires to align and connect.

Various other modifications, adaptations, and alternate configurations are of course possible in light of the teachings of the present invention presented above. Therefore, it should be understood at this time that, within the scope of the appended Claims, the invention may be practiced otherwise than as specifically described herein.

What is claimed is:

1. An implantable medical system for implantation within the body of a patient comprising:
an implanted device including an implant casing and a long range telemetry sub-system housed therein;
an implantable lead operationally coupled to said implanted device; and
an antenna coupled to said implant casing to extend therefrom, said antenna being operationally coupled to said long range telemetry sub-system of said implanted device for wireless bi-directional communication therethrough between said long range telemetry sub-system and predetermined external equipment disposed outside the body of the patient.

2. The implantable medical system of claim 1 further comprising a connecting cable coupled between said implanted device and said implantable lead, said connecting cable including a plurality of conductors, a first of said conductors terminating at a free end to form said antenna.

3. The implantable medical system of claim 1, wherein said implanted device includes a header attached to said implant casing, said implantable lead being coupled to said implant casing through said header.

4. The implantable medical system of claim 3, wherein said antenna extends from said header and terminates at a free end disposed outside said implanted device.

5. The implantable medical system of claim 2, wherein said implantable lead includes at least one electrode, a second of said conductors of said connecting cable being coupled to said electrode.

6. The implantable medical system of claim 1, wherein said implanted device includes a window formed of a non-conductive material in a side wall of said implant casing, said antenna being disposed adjacent said window.

7. The implantable medical system of claim 6, wherein said antenna is disposed on said window to extend along an outer surface thereof.

8. The implantable medical system of claim 6, wherein said antenna is disposed on said window to extend along an inner surface thereof.

9. The implantable medical system of claim 6, wherein said antenna is disposed within said window to be at least partially embedded therein.

10. The implantable medical system of claim 1, further comprising patient alerting means operationally coupled to said implanted device.

11. The implantable medical system of claim 1, wherein said implantable lead is configured for cardiac implantation.

12. The implantable medical system of claim 1, wherein said implantable lead is configured for neurostimulation implantation.

13. The implantable medical system of claim 1, wherein the range of the data communication between said implanted device and said external equipment is greater than 1 foot.

14. The implantable medical system of claim 1, wherein the range of the data communication between said implanted device and said external equipment is greater than 1 meter.

15. The implantable medical system of claim 1, wherein said implanted device includes device circuitry housed in said implant casing, at least portion of said device circuitry being selected from the group consisting of: electrical stimulation circuitry, pacemaker circuitry, and defibrillator circuitry.

16. The implantable medical system of claim 1, wherein said long range telemetry sub-system is switched between ON and OFF states during a preset period of time in a cyclical manner.
